(19) United States
(12) Patent Application Publication
(10) Pub. No.: US 2025/0194721 A1
(43) Pub. Date: Jun. 19, 2025

(54) Arm Sleeve with Pocket

(71) Applicant: Andrea Lyss, Blacklick, OH (US)

(72) Inventor: Andrea Lyss, Blacklick, OH (US)

(21) Appl. No.: 18/544,608

(22) Filed: Dec. 19, 2023

(51) Int. Cl. A41D 27/10; A61F 13/10

(52) U.S. Cl. CPC A41D 27/10; A61F 13/10

(57) ABSTRACT

This invention is a compression arm sleeve that includes a small pocket to be placed in the inner forearm.

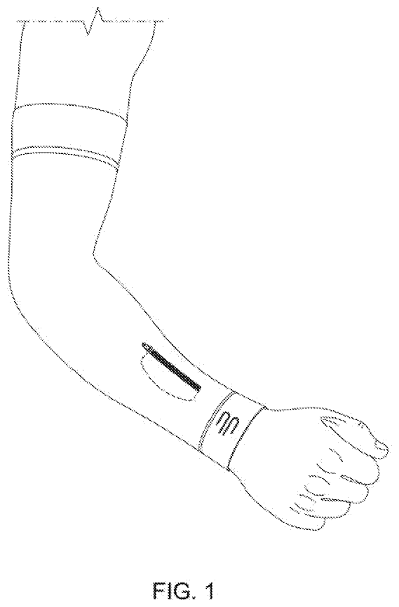

### FIELD OF INVENTION

The invention can be in the field of fashion, athletics, athletic leisurewear.

### DESCRIPTION OF THE RELATED ART

In my research, I have not yet found a compression arm sleeve that includes a pocket. I thought of this invention when I was training for a marathon and would get really frustrated when I had to search for something small in my fanny pack.

### BRIEF SUMMARY

This invention is a compression arm sleeve that includes a small pocket to be placed in the inner forearm.

Drawings will be attached to the application.

### DETAILED DESCRIPTION

This invention is a compression arm sleeve with a pocket. A small pocket, about 2 inches in length, that could fit anything, and not limited to, the size of a key. The invention will most likely be used by athletes, and can be used by anyone for any purpose. Examples: runners who keep snacks in the pockets, a young woman going to a festival that wants to keep something valuable or protective in the pocket. The arm sleeve itself will be light, made of nylon and spandex.

1. claim Listing.

2. The invention is a compression arm band with the pocket, The pocket is the invention, as I have not found any arm sleeves with such pockets, The pocket can be any size.
